(19) United States
(12) Patent Application Publication
(10) Pub. No.: US 2013/0274131 A1
(43) Pub. Date: Oct. 17, 2013

(54) Advanced Reverse-phase Magnetic Immunoassay

(71) Applicant: The Board of Trustees of the Leland Stanford Junior University, (US)

(72) Inventors: Jung-Rok Lee, Stanford, CA (US); Richard Gaster, Mountain View, CA (US); Shan X. Wang, Portola Valley, CA (US)

(21) Appl. No.: 13/861,120

(22) Filed: Apr. 11, 2013

Related U.S. Application Data
(60) Provisional application No. 61/622,872, filed on Apr. 11, 2012.

(51) Int. Cl. G01N 33/543

(52) U.S. Cl. CPC G01N 33/54306
USPC 506/9

(57) ABSTRACT

A magnetic assay is provided where capture probes are disposed on the sensor array as capture probe spots that overlap with the sensor elements. The capture probes on the sensor elements can be the same or they can be different. Two or more sample solutions are also disposed on the sensor array as sample spots that overlap with the sensor elements. Targets in the sample solutions can bind to the capture probes to provide immobilized targets. Magnetically labeled probes capable of binding to targets are provided to the assay, and the resulting assay signal is from immobilized magnetically labeled probes at the sensor elements.

### CROSS REFERENCE TO RELATED APPLICATIONS

This application claims the benefit of U.S. provisional patent application 61/622,872, filed on Apr. 11, 2012, and hereby incorporated by reference in its entirety.

### GOVERNMENT SPONSORSHIP

This invention was made with Government support under contract numbers CA143907 and CA151459 awarded by the National Institutes of Health. The Government has certain rights in this invention.

### FIELD OF THE INVENTION

This invention relates to magnetic assays.

### BACKGROUND

Assays are widely used for various chemical, biological and biochemical applications. Conventional assays can be categorized as forward phase or reverse phase. In a typical forward phase assay, a sensor array is employed, where each sensor array element has a different antibody immobilized at its surface. A sample solution possibly containing targets that can bind to the antibodies on the sensor elements is provided to the device (same sample solution at each sensor element). Labeled probes bind to immobilized targets (if any) to provide the assay signal. This provides measurements of multiple targets in a single sample solution.

In a typical reverse phase assay, the samples are immobilized on the sensor array (different sample on each sensor element). A solution containing labeled probes is provided to the sensor array (same probe solution at each sensor element). Labeled probes bind to immobilized targets in the samples to provide the assay signal. This provides measurements of a single target in multiple samples.

However, this present state of the art is unsatisfactory in some respects. In particular, the sensitivity of conventional reverse-phase assays tends to be low. Accordingly, it would be an advance in the art to address this limitation.

### SUMMARY

The present approach provides a magnetic assay where capture probes are disposed on the sensor array as capture probe spots that overlap with the sensor elements. The capture probes on the sensor elements can be the same or they can be different. Two or more sample solutions are also disposed on the sensor array as sample spots that overlap with the sensor elements. Targets in the sample solutions can bind to the capture probes to provide immobilized targets. Magnetically labeled probes capable of binding to targets are provided to the assay, and the resulting assay signal is from immobilized magnetically labeled probes at the sensor elements. Assays according to these principles are suitable for use in any biological, chemical or biochemical situation where an assay is applicable.

One significant application of this work is in cancer research. In the past decade, there have been considerable efforts in finding cancer biomarkers in terms of tumor antigens such as prostate-specific antigen (PSA), Carcino-Embryonic antigen (CEA), cancer antigen CA125, and so on as well as in utilizing them for early detection. Some other proteins such as hormones and enzymes have been used as markers for cancer detection or monitoring. Recent studies show that detection of autoantibody could be a better diagnostic method with higher sensitivity and specificity for early detection. In addition, the need for measuring multiple samples over time from a single patient during treatment have been growing in order to understand cancer dynamics and pharmacodynamics better.

With the present approach, many different molecules such as cytokines and antibodies in multiple samples can be measured simultaneously with a single device. A panel of multiple types of biomarkers provides us with a better insight into the cancer, and can provide higher sensitivity and specificity of diagnostics. Moreover, consumption of small volume of samples allows us to conduct the experiments where the volume of sample is limited or frequent bleedings are required in a single mouse, which cannot be measured with existing techniques. The unique features of this technique will enable scientists to gain more biological information with fewer devices and less sample volume.

### BRIEF DESCRIPTION OF THE DRAWINGS

FIG. 1A-B shows examples relating to embodiments of the invention.

FIG. 2 shows an example of an assay of multiple targets and multiple samples according to principles of the invention.

FIG. 3 provides diagrams showing the significance of variable capture probe spot and sample spot overlap.

FIGS. 4A-B show examples of how magnetically labeled probes can be provided to the sensor array.

FIG. 5 shows several examples of various capture probes, target species, and labeled probes.

FIGS. 6A-B show an exemplary sequence resulting in an immobilized labeled probe at a sensor element.

FIGS. 7A-D show another exemplary sequence resulting in an immobilized labeled probe at a sensor element.

FIGS. 8A-C show a further exemplary sequence resulting in an immobilized labeled probe at a sensor element.

FIGS. 9A-C show an exemplary approach for providing a spot pattern of immobilized capture probes.

FIG. 10A schematically shows the capture probe arrangement in a first experiment.

FIG. 10B schematically shows the sample spot arrangement in the first experiment.

FIGS. 11A-F are results from the first experiment.

FIGS. 12A-C are results from a second experiment.

### DETAILED DESCRIPTION

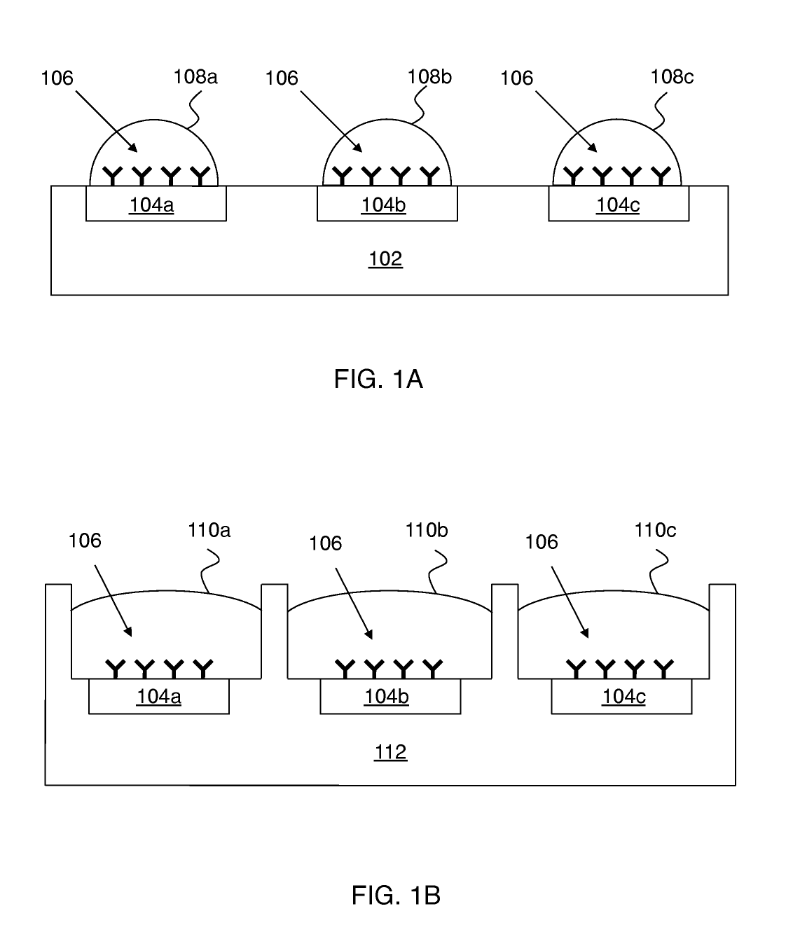

FIG. 1A-B shows examples relating to embodiments of the invention in side views. In the example of FIG. 1A, a substrate 102 includes magnetic sensor array elements 104a, 104b, and 104c. Capture probes 106 are immobilized at each of the sensor elements in a capture probe spot pattern. Samples 108a, 108b, and 108c are provided to the sensor elements in a sample spot pattern. Practice of the invention does not depend critically on how these spot patterns are formed. In the example of FIG. 1A, surface tension is relied upon to define the sample spots after they are deposited. In the example of FIG. 1B, structural dividers as part of substrate 112 are used to define the sample spots after they are deposited. More generally, the capture probes can be delivered by any method, including but not limited to: spotting, microfluidic delivery, confinement of capture probes in well structures, gel-based delivery, paper-based delivery, and imprinting. Similarly, sample delivery to provide the sample spot pattern can be performed by any method, including but not limited to: spotting, microfluidic delivery, confinement of samples in well structures, gel-based delivery, paper-based delivery, and imprinting.

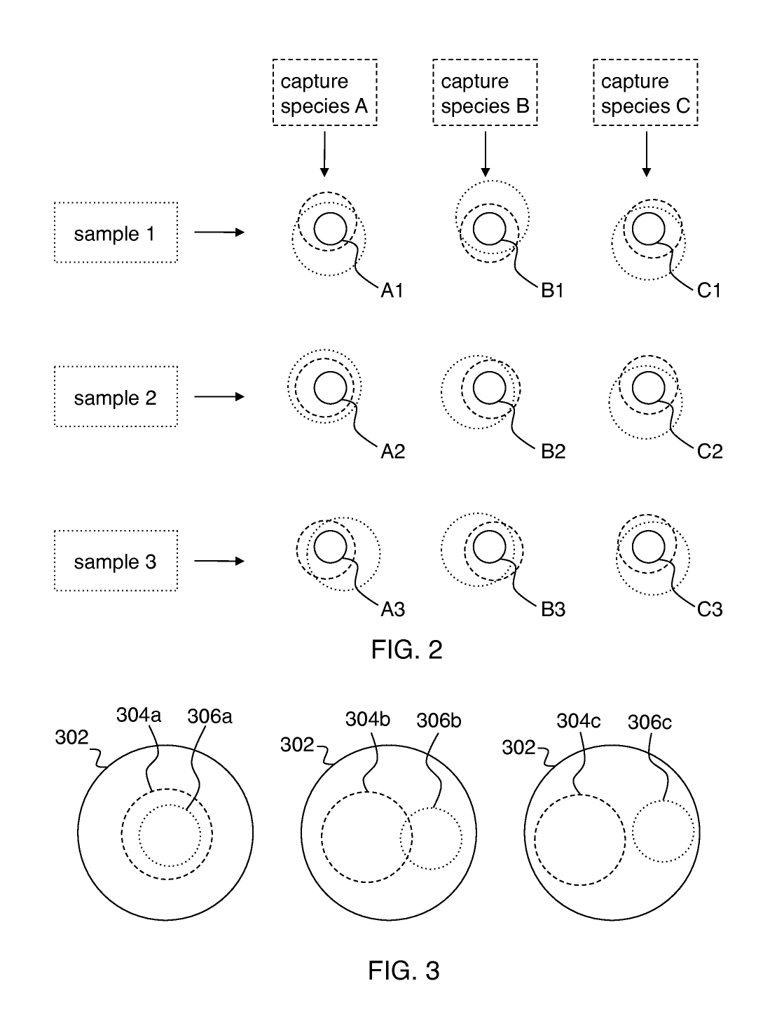

The example of FIGS. 1A-B show the same capture probes at each sensor array element. It is also possible for different capture probes to be used at different sensors in the array. FIG. 2 shows an example, where now a top view is employed, as opposed to the side views of FIGS. 1A-B. The sensor array of FIG. 2 has nine elements arranged in a 3×3 array. Each column of the array has a different capture probe immobilized at its sensor elements (dashed lines show the capture probe spots). Each row of the array has a different sample provided to it (dotted lines show the sample spots). As a result, each sensor acts as described by its reference (e.g., sensor A1 relates to sample 1 and capture species A, sensor C2 relates to sample 2 and capture species C, etc.).

An exemplary method for performing a biological or chemical assay according to principles of the invention includes the following steps:

a) providing an array of magnetic sensors (2 or more sensor elements);

b) immobilizing capture probes at the sensor elements, where the capture probes are configured as discrete capture probe spots, each capture probe spot overlapping with a corresponding one of the sensor elements;

c) providing sample solutions to the sensor elements. The sample solutions include two or more distinct samples that are provided to different sensor elements. The sample solutions are configured as discrete sample spots, each sample spot overlapping with a corresponding one of the sensor elements. The sample solutions can include target species that can bind to the capture probes to become immobilized targets.

d) providing one or more magnetically labeled probes to the sensor elements capable of binding to the targets. Practice of the invention does not depend critically on details of how the labeling is performed. Several alternative labeling methods are described in examples below.

e) providing signal measurements from the sensor elements responsive to immobilized magnetically labeled probes as the assay output.

The two or more distinct samples can be from multiple subjects and/or they can be from the same subject at different times. Practice of the invention does not depend critically on the kind of magnetic sensor employed. Suitable magnetic sensors include, but are not limited to: spin valve sensors, magnetic tunnel junction sensors, and Hall-effect sensors. The incubation time for the sample solutions on the immobilized capture probes can range from 5 minutes to 24 hours depending on the application.

From FIG. 2, it is apparent that the present approach entails forming discrete spots of both capture probes and samples. We have unexpectedly found that this double spotting approach can work well in practice, and in particular, that the undesirable increase in signal variability that one would expect from double spotting can be avoided.

FIG. 3 shows an example of the kind of issues that can arise when double spotting. In these examples, a detector active area 302, a sample spot (306a, 306b, or 306c) and a capture probe spot (304a, 304b, or 304c) overlap. The detected signal is only from the area where all three spots overlap. Thus, on the left side of FIG. 3, the assay signal is maximized (full overlap of all spots). In the middle of FIG. 3, the assay signal is much weaker (spots 304b and 306b have poor overlap). On the right side of FIG. 3, there would be no assay signal at all, because spots 304c and 306c do not overlap. In practice, it is not possible to deposit capture probe spots and sample spots with perfect precision, so some variability in capture probe and sample spot position must be expected. In cases such as shown on FIG. 3, such spot position variability can fatally compromise the assay by introducing spurious variability in signal as a result of spot overlap variation, as opposed to the real assay results. In other words, variable spot overlap as shown on FIG. 3 is a significant source of experimental noise for the assay.

In view of this issue relating to spot overlap, it is preferred for the active area of the sensor elements to be smaller than the spot size of the capture probes and also smaller than the spot size of the sample spots. Such a configuration is shown on FIG. 2. Here is it apparent that the variable overlap of capture spots and signal spots (as shown on FIG. 2) will not affect the assay results, because the effective area in all cases is that of the sensor element. From this, it is apparent that the problem in the example of FIG. 3 was that the assumed sensor active area was larger than the sample spot size and capture probe spot size, so ensuring perfect overlap between the sensor and the sample/capture probes (as shown on FIG. 3) did not ensure a consistent level of three way overlap. Magnetic sensors are particularly suitable for this kind of assay, since they naturally provide a small active area for each element of the sensor array. Other commonly employed sensor technologies (e.g., optical detection) tend to undesirably provide larger sensor element active area.

For completeness, it is noted that spot position variability can still affect assay results even in the preferred configuration of FIG. 2 (i.e., small sensor element active area). For example, the overlap between the sample spot and/or capture probe spot and the sensor could be partial rather than complete. In such cases, a variable level of spot overlap would contribute to experimental noise in the assay. However, in practice, a significant improvement is usually realized, since it is typically much easier to provide alignment of sample spots and capture probe spots to the sensor elements than to align sample spots and capture probe spots to each other.

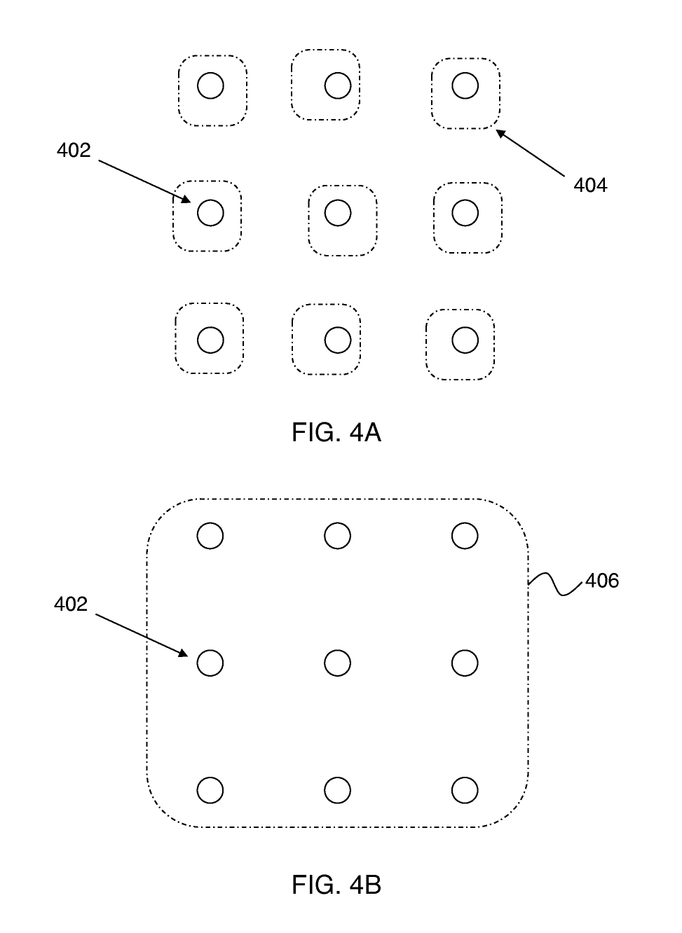

FIGS. 4A-B show examples of how magnetically labeled probes can be provided to the sensor array. In the example of FIG. 4A, the magnetically labeled probes are also spotted in a labeled probe spot pattern 404 that corresponds to sensor array 402. Such a spot pattern can be used to provide two or more distinct magnetic probe species to different sensor elements. In the example of FIG. 4B, a single magnetic probe solution 406 is provided to all sensor elements. Incubation times for the magnetically labeled probes can be from 5 minutes to 2 hours depending on the application.

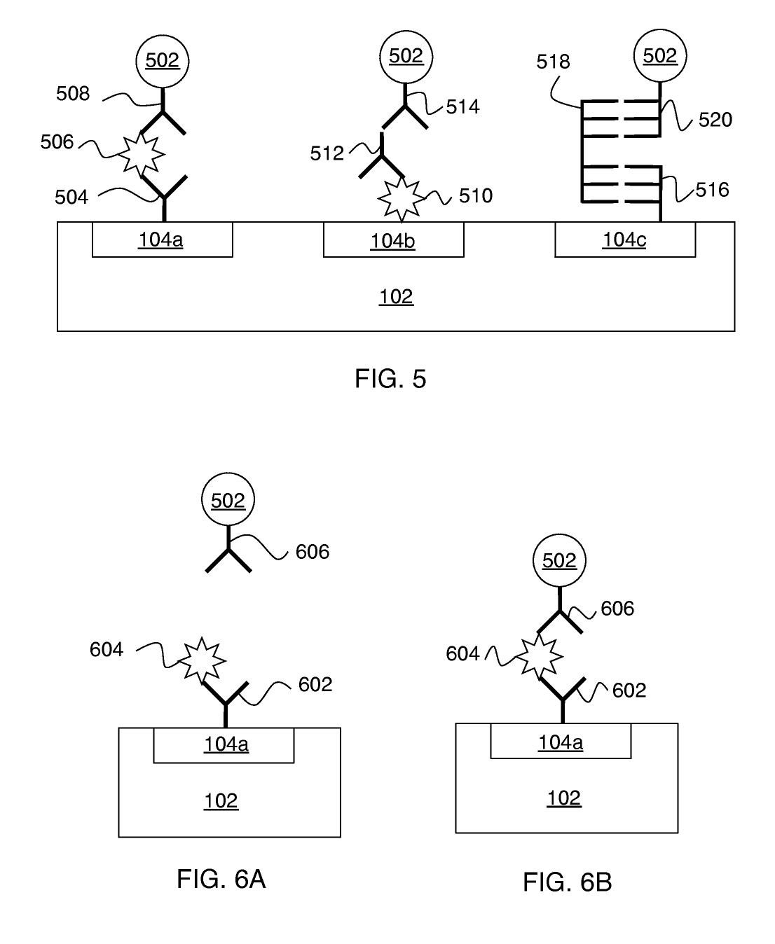

Practice of the invention does not depend critically on which chemical species are designated as capture probe, target and label probe. All combinations that can provide immobilized magnetically labeled probes on a sensor element to provide an assay signal can be used. FIG. 5 shows several examples of various capture probes, target species, and labeled probes. More specifically, the left part of FIG. 5 has capture species 504 being an antibody, target 506 being an antigen, and label probe 508 being a detection antibody bound to a magnetic label 502. The center part of FIG. 5 has capture species 510 being an antigen, target 512 being a serum antibody, and label probe 514 being a detection antibody bound to magnetic label 502. The right part of FIG. 5 has capture species 516 being an oligonucleotide, target 518 also being an oligonucleotide, and label probe 520 being a detection oligonucleotide bound to magnetic label 502.

An antibody includes two parts: Fab and Fc. Fab is the binding site for the target antigen, and the Fc part interacts with immune cells to trigger immune responses. It is convenient to refer to the Fab part of an antibody as a fragment-antigen binding fragment (FAB-fragment).

A “capture probe” is defined to be any species that can be immobilized onto a surface and which is capable of selectively binding a target species. Examples include, but are not limited to: oligonucleotides, peptides, antibodies, antigens, fragment-antigen binding fragments (FAB-fragments), aptamers, diabodies, DNA sequences, and RNA sequences.

A “target species” is defined to be any chemical or biochemical species of interest, including but not limited to: oligonucleotides, peptides, antibodies, antigens, fragment-antigen binding fragments (FAB-fragments), aptamers, diabodies, DNA sequences, and RNA sequences.

Practice of the invention does not depend critically on details of the magnetic labeling, and in particular, any sequence of events that leads to immobilized magnetically labeled probe-target complexes at the sensor elements as the assay signal can be employed.

FIGS. 6A-B show an exemplary sequence resulting in an immobilized labeled probe at a sensor element. In this example, FIG. 6A shows an immobilized target 604 bound to capture species 602 with a magnetically labeled probe 606 (including magnetic label 502) nearby in solution. FIG. 6B shows the result of this magnetically labeled probe binding to the immobilized target. This sequence can be ensured by washing the sample solutions off the sensor elements prior to providing the magnetically labeled probes, since the only targets that will remain after the washing step are the ones that have been bound by capture probes.

The example of FIGS. 6A-B shows another feature that is commonly used in practice. In this example, the magnetically labeled probe is provided as a complex of probe 606 bound to magnetic label 502. This can be regarded as the result of binding magnetic labels to probe species, followed by providing the magnetically labeled probe species to the targets.

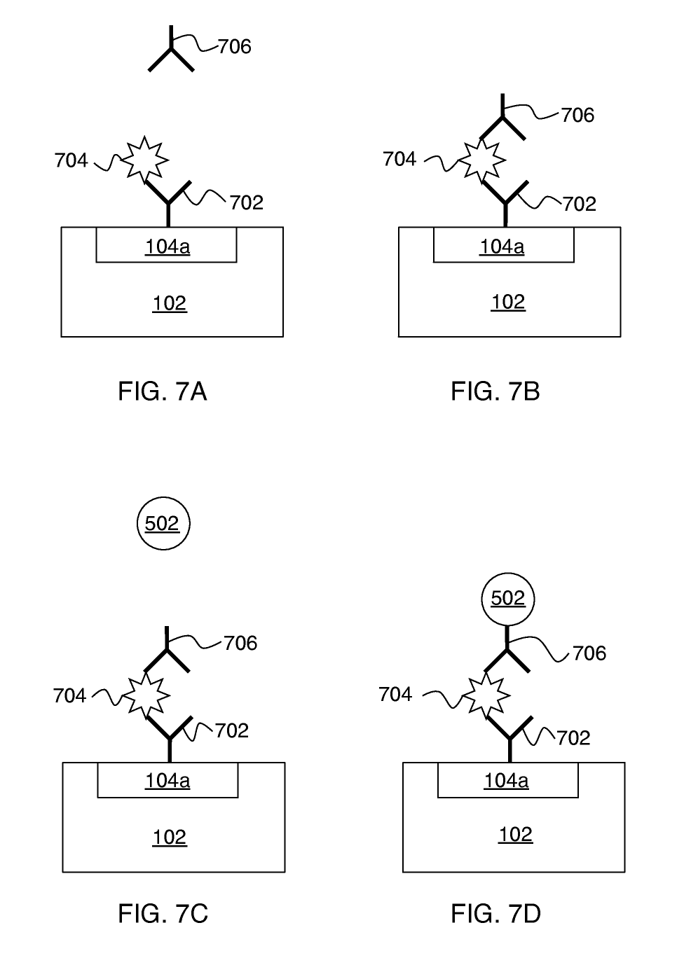

An alternative labeling sequence is shown on FIGS. 7A-D. In this example, FIG. 7A shows providing a probe species 706 to sensor element 104a (which has a target 704 immobilized by capture probe 702). FIG. 7B shows binding of probe 706 to target 704. FIG. 7C shows providing magnetic label 502, and FIG. 7D shows binding of magnetic label 502 to probe 706.

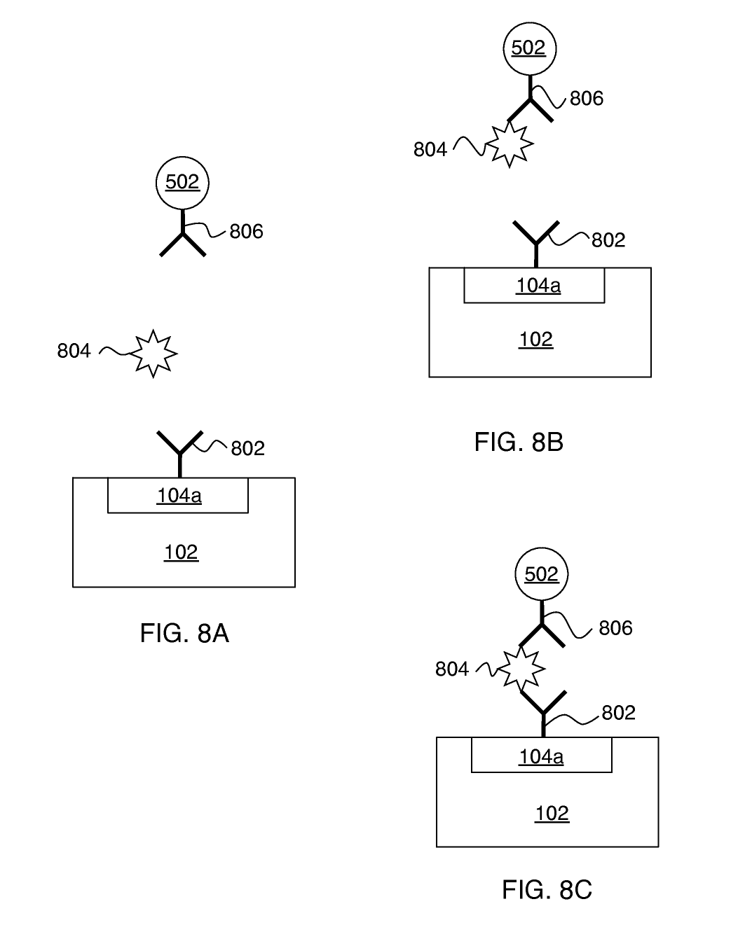

FIGS. 8A-C show a further exemplary sequence resulting in an immobilized labeled probe at a sensor element. In this example, the magnetically labeled probes (a complex of probe 806 and magnetic label 502) are provided to the sensor elements while the sample solutions are present. Thus, FIG. 8A shows both target 804 and the magnetically labeled probe in solution. FIG. 8B shows binding of target 804 to the magnetically labeled probe in solution, and FIG. 8c shows binding of the target-label complex to capture probe 802.

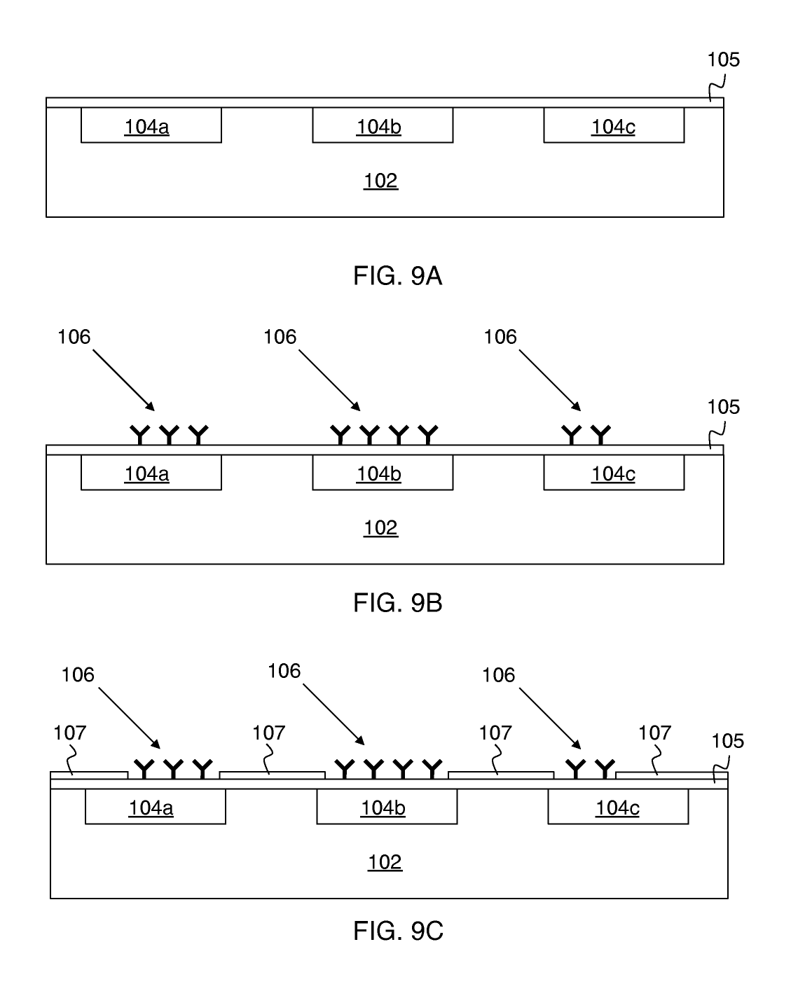

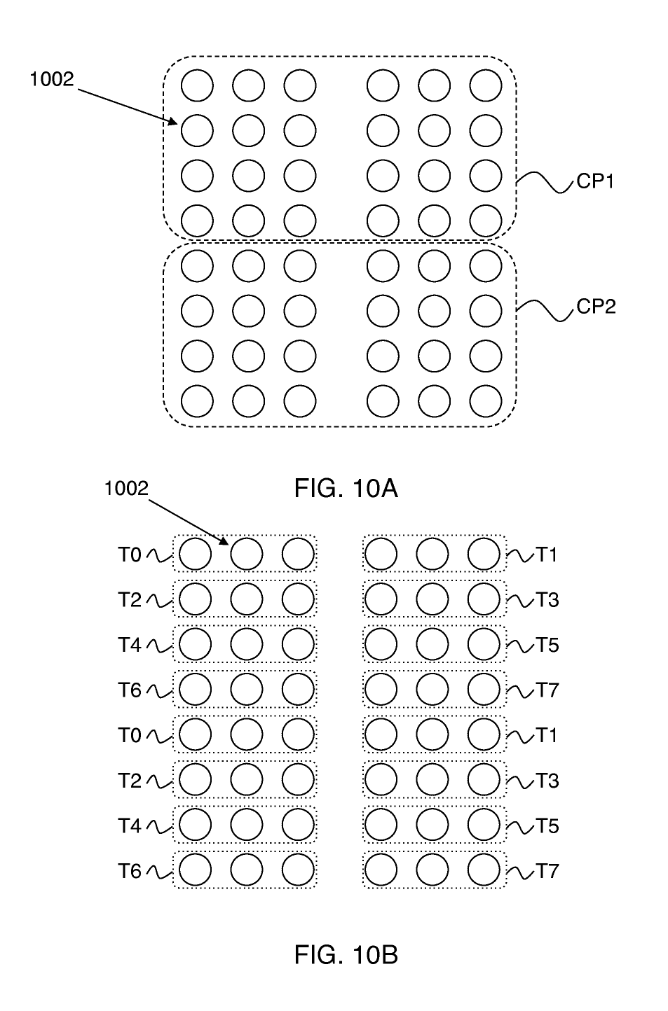

FIGS. 9A-C show an exemplary approach for providing a spot pattern of immobilized capture probes. In this example, substrate 102 including sensor array elements 104a, 104b, and 104c has its surface treated to provide binding of the capture probes. Schematically, this surface treatment is shown as a thin layer 105 on FIG. 9A. In practice, such surface treatment may or may not result in the formation of a separate layer. For example, if polyethylenimine (PEI) is used for surface treatment, a very thin layer (possibly a monolayer) of PEI is formed on the surface, which would be 105 here. FIG. 9B shows deposition of the capture probe spot pattern, where incomplete coverage of the sensor elements by the capture probes is shown (this can easily occur in practice). FIG. 9C shows the result of applying a blocking layer 107 to parts of the treated surface not covered by capture probes. The purpose of this blocking layer is to prevent binding of other species to the treated surface.

Although it would be ideal for treatment layer 105 to specifically bind capture probes and nothing else, practical realization of the surface treatment is not likely to provide perfect specificity. Thus, the use of a blocking layer 107 compensates for this lack of perfect specificity. Non-specific binding of magnetically labeled probes to treated surface 105 is particularly undesirable, since it would give false positives (signal with no target) in assays. In order to block the surface, a blocking buffer of 1% Bovine Serum Albumin (BSA) in Phosphate Buffered Saline (PBS) can be added to the reaction well for one hour. The concentration of BSA and incubation time might vary depending on applications. Other blocking materials can be used. For example, milk fat can be employed.

Depending on the capture probes being used, certain chemicals can be used for treatment to specifically target the capture probes. The specificity of this chemistry might be broader than that of capture probes to target species in the sample, but we need to design this layer to have good bindings to the capture probes. In some work, we use NHS-EDC and some other polymers to link the amine group on the capture probes to the surface. Here NHS is an abbreviation of N-Hydroxysuccinimide, and EDC is an abbreviation of 1-Ethyl-3-(3-dimethylaminopropyl)carbodiimide

A cationic polymer such as polyethyleneimine (PEI) can be used to nonspecifically bind charged antibodies to the sensor surface via physisorption. Alternatively, a covalent chemistry can be used utilizing free amines or free thiol groups on the capture probes.

The following description relates to two experimental demonstrations of principles of the invention. In the first experiment, samples from different times were simultaneously assayed using two different capture probes. In the second experiment, C-reactive protein was simultaneously measured in multiple samples with high sensitivity.

In the first experiment, the immune response to luciferase molecules in mice was measured. At day 0, luciferase-expressing tumor cells were injected into six mice. Three mice (C1, C2, and C3) were used as controls, and the other three mice (L1, L2 and L3) were treated by an injection of LPS (lipopolysaccharides). A total of 8 samples per mouse was obtained, starting at day 0 and having an interval of 3 days between samples. The day 0 samples were drawn prior to tumor cell injection. The samples were 5 μL serum samples diluted 5× with PBS. Thus, the available volume was 25 μL, of which about 10 μL was used for spotting the sample onto the sensor array

FIG. 10A schematically shows the capture probe arrangement in a first experiment. Here sensor array 1002 is divided into two regions. Region CP1 is for the first capture probes, which were LPS, and region CP2 is for the second capture probes, which were probes binding to various isotypes of immunoglobulins (i.e., IgM, IgG1, IgG2a, IgG2b, and IgG3). For any one experiment, all of the probes in CP2 would bind to the same target (e.g., IgM). The experiment was repeated with different CP2 probes to cover the various immune system factors.

FIG. 10B schematically shows the sample spot arrangement in the first experiment. Here T0, T1, . . . T8 refer to the sample times. All these samples are from the same mouse. Thus, a single run of the assay simultaneously gives the CP1 and CP2 assay of a mouse at all of its sample times. The small sample volume needed by the present approach is a significant advantage relative to conventional assays that can require much more sample volume. This experimental protocol would not be possible if the sample volumes had to be much larger. If one is going to take blood from a mouse every three days, the blood draws need to be small.

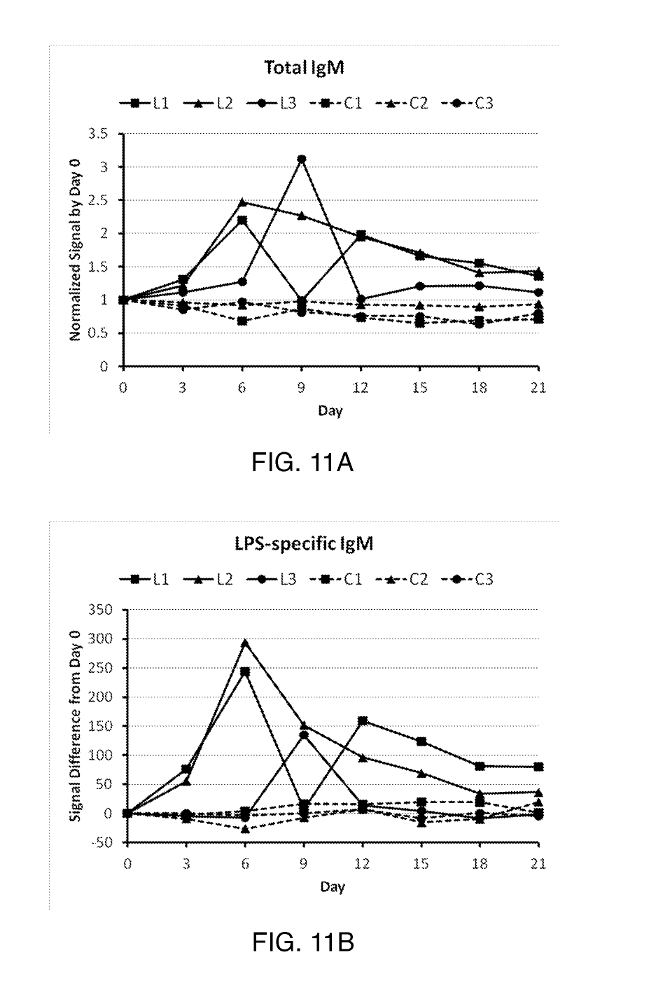

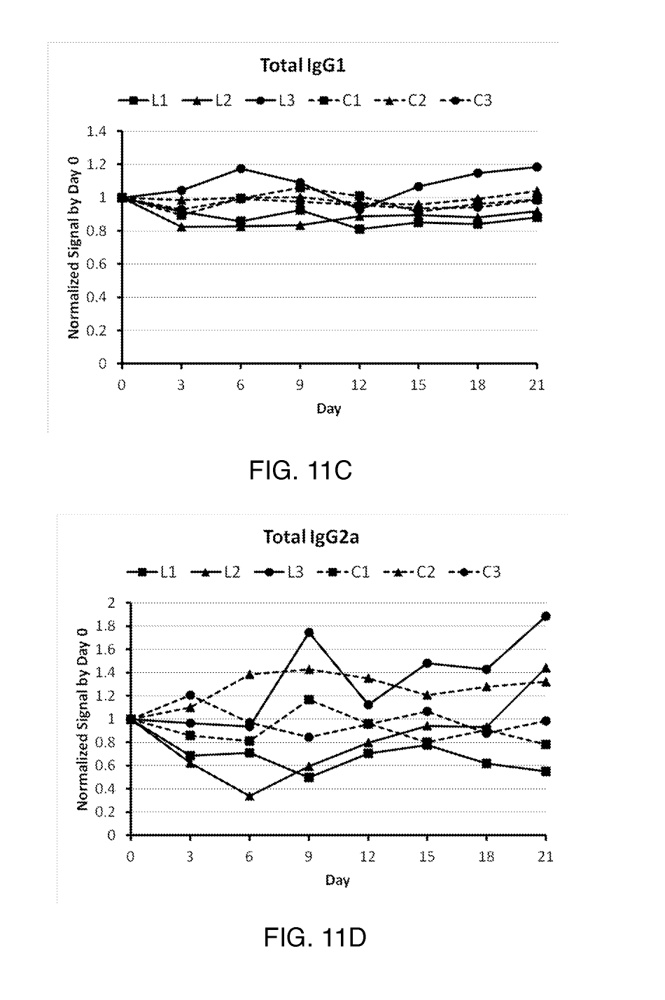

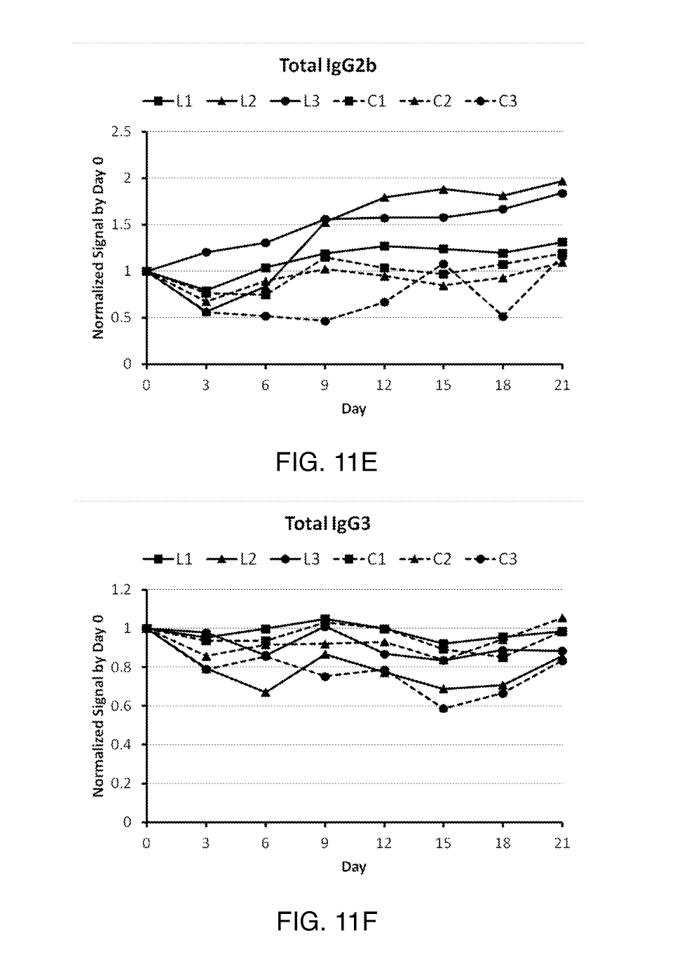

FIG. 11A shows results for total IgM. FIG. 11B shows results for LPS-specific IgM. FIG. 11C shows results for total IgG1. FIG. 11D shows results for total IgG2a. FIG. 11E shows results for total IgG2b. FIG. 11F shows results for total IgG3. IgM can be LPS specific if any of its five Fab regions recognize LPS, as will occur if the immune system develops IgM against LPS as part of an immune response.

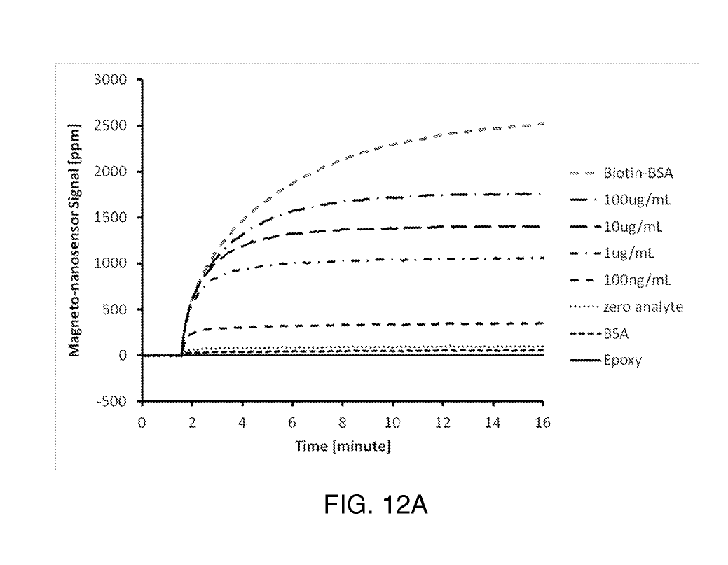

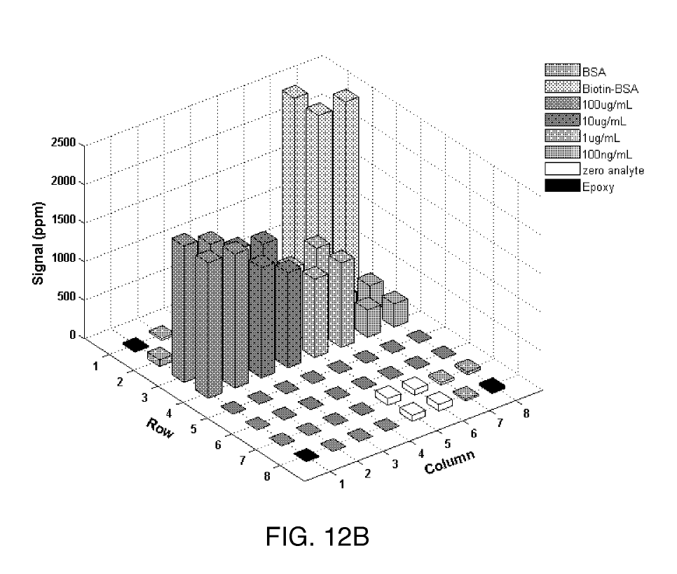

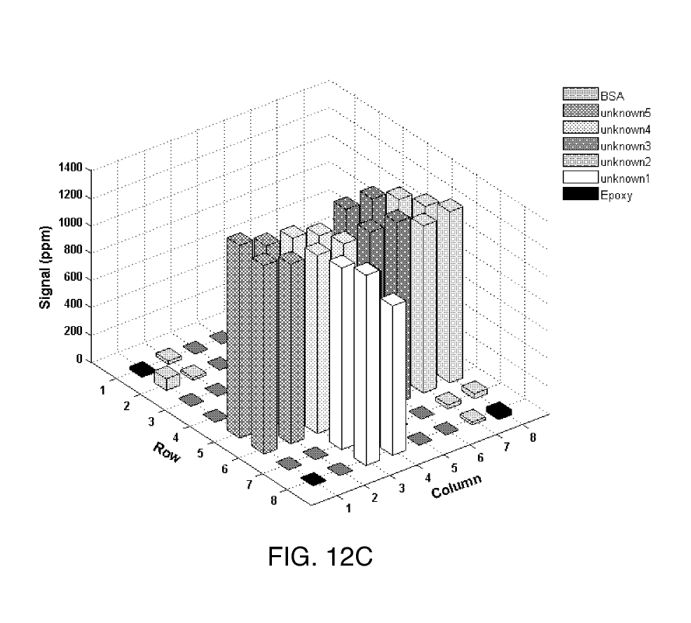

FIGS. 12A-C are results from a second experiment. With an 8×8 array of giant magneto-resistance (GMR) sensors, we are able to demonstrate the ability of measuring C-reactive Protein (CRP) in multiple samples. First, the sensors were functionalized with capture probes (i.e., a capture antibody for CRP), while some of them were covered with Bovine Serum Albumin (BSA), BSA conjugated with biotin molecules, or a thick passivation layer, which were used as biological negative controls, positive controls, and electrical negative controls, respectively. After incubating the array with the capture antibody for one day and washing it, different samples, with known or unknown analyte concentrations, were spotted on different individual sensors locally with a piezo-capillary dispenser. The incubation and washing step was repeated. Then, detection antibody of CRP was introduced into the reaction well, and labeled with magnetic nanoparticles.

The samples with known but different concentrations, so-called standard samples, were spotted on the sensors with four repeats. The averaged binding curves are shown in FIG. 12A. The onset of the curves at about 2 minutes is the time when the magnetic nanoparticles are introduced into the reaction well. Therefore, these binding curves indicate the bindings between the nanoparticles and the sandwiched protein assemblies (FIG. 5), more specifically streptavidin on the particle and biotin on the detection probe. Data was taken for sample CRP concentrations of 100 μg/mL, 10 μg/mL, 1 μg/mL, and 100 ng/mL. The zero analyte sample was a buffer solution that did not contain any CRP. FIG. 12B shows the signals from each sensor at 14 minutes when the reaction reaches equilibrium so the signal remains constant.

As proof of principle, we have tested serum from mice with lymphoma. The samples with unknown CRP concentrations were also spotted on the same array, and measured at the same time when the results shown in FIGS. 12A-B were obtained. The gray-colored sensors shown in FIG. 12B were the sensors with the unknown samples and their signals are not displayed on FIG. 12B in order to show the signals from the standard samples more clearly. The results from the sensors with the unknown samples are shown in FIG. 12C. Each of the unknown samples is spotted on 4 different sensors. These signals can be converted into concentrations by comparing these signals with the signals from standard samples.

1. A method for performing a biological or chemical assay, the method comprising:
providing an array of magnetic sensors including two or more sensor elements;
immobilizing capture probes on the sensor elements, wherein the immobilized capture probes are configured as discrete capture probe spots, each capture probe spot overlapping with a corresponding one of the sensor elements;
providing sample solutions on the sensor elements, wherein the sample solutions comprise two or more distinct individual samples disposed on different sensor elements, wherein target species in the sample solutions may bind to immobilized capture probes to become immobilized targets; and wherein the sample solutions are configured as discrete sample spots, each sample spot overlapping with a corresponding one of the sensor elements;
providing one or more magnetically labeled probes to the sensor elements capable of binding to the targets; and
providing signal measurements from the sensor elements responsive to immobilized magnetically labeled probes as an assay output.

2. The method of claim 1, wherein an active area of at least one of the sensor elements is smaller than a spot size of the capture probes and smaller than a spot size of the sample solutions.

3. The method of claim 1, wherein the immobilizing capture probes comprises:
treating a surface of the sensor elements to provide a treated surface capable of binding at least the capture probes;
disposing the capture probes on the treated surface to provide the immobilized capture probes; and
blocking parts of the treated surface not covered by the immobilized capture probes to prevent binding of species other than the capture probes.

4. The method of claim 1, wherein the immobilizing capture probes comprises a capture probe delivery method selected from the group consisting of: spotting, microfluidic delivery, confinement of capture probes in well structures, gel-based delivery, paper-based delivery, and imprinting.

5. The method of claim 1, wherein the immobilized capture probes comprise one or more distinct capture species disposed on different sensor elements.

6. The method of claim 1, wherein the two or more distinct individual samples are samples from multiple subjects.

7. The method of claim 1, wherein the two or more distinct individual samples are samples from a single subject at different times.

8. The method of claim 1, wherein the providing sample solutions comprises a sample delivery method selected from the group consisting of: spotting, microfluidic delivery, confinement of samples in well structures, gel-based delivery, paper-based delivery, and imprinting.

9. The method of claim 1, wherein the magnetically labeled probes comprise two or more distinct magnetic probe species provided to different sensor elements.

10. The method of claim 1, wherein the magnetically labeled probes comprise a single magnetic probe solution provided to the sensor elements.

11. The method of claim 1, further comprising washing the sample solutions off the sensor elements prior to the providing one or more magnetically labeled probes, whereby the magnetically labeled probes bind to immobilized targets.

12. The method of claim 1, wherein the magnetically labeled probes bind to the targets in the sample solutions to provide labeled targets, and wherein the labeled targets bind to the capture probes to provide the immobilized magnetically labeled probes.

13. The method of claim 1, wherein the providing one or more magnetically labeled probes to the sensor elements comprises:
binding magnetic labels to probe species followed by providing magnetically labeled probe species to the targets.

14. The method of claim 1, wherein the providing one or more magnetically labeled probes to the sensor elements comprises:
providing probe species to the sensor elements followed by providing magnetic labels to the sensor elements, wherein the magnetic labels are capable of binding to the probe species.

15. The method of claim 1, wherein the sensor elements are selected from the group consisting of: spin valve sensors, magnetic tunnel junction sensors, and Hall-effect sensors.
